(19) United States
(12) Patent Application Publication
(10) Pub. No.: US 2007/0123982 A1
(43) Pub. Date: May 31, 2007

(54) Intra-ocular lens

(75) Inventors: Michael Yablonski, Vestal, NY (US); David Peters, Whitney Point, NY (US)

Correspondence Address: SEED INTELLECTUAL PROPERTY LAW GROUP PLLC, 701 FIFTH AVE, SUITE 5400, SEATTLE, WA, 98104, US

(73) Assignee: Judith Yablonski, Eugene, OR (US)

(21) Appl. No.: 11/509,300

(22) Filed: Aug. 23, 2006

Related U.S. Application Data
(63) Continuation of application No. 10/677,651, filed on Sep. 22, 2003, now abandoned.
Application No. 10/677,651 is a continuation-in-part of application No. 10/012,185, filed on Nov. 6, 2001, now abandoned.

(51) Int. Cl. A61F 2/16

(52) U.S. Cl. 623/6.37; 623/6.46

(57) ABSTRACT

An accommodating, intra-ocular lens that is gravity activated, and is constructed from materials that provide a specific gravity greater than that of the aqueous humor. The lens is constructed with a number of haptics: two horizontal haptics and possibly an inferior haptic which prevents the heavier-than-aqueous-fluid lens from de-centering inferiorly. The haptics are disposed within the lens in an articulated manner that permits movement of the lens between forward and rearward positions.

### RELATED PATENT APPLICATION

This patent application is a continuation-in-part of copending U.S. patent application Ser. No. 10/012,185, filed Nov. 6, 2001.

### FIELD OF THE INVENTION

The invention relates to cataract surgery methods and apparatuses and, more particularly, to an improved gravity-activated intra-ocular lens that is inserted in the eye during cataract surgery. The intra-ocular lens is designed to change position within the eye in order to change the focal point of the eye to provide for both near vision and far vision.

### BACKGROUND OF THE INVENTION

U.S. Pat. No. 5,522,891, granted to Klaas f or INTRAOCULAR LENS on Jun. 4, 1996, illustrates an intra-ocular lens that is gravity activated to change the focal point to provide both near vision and far vision. The lens moves forward in the eye to permit focusing on a near object, and back to its original posterior position to permit focusing on a distant object. The problem with the subject intra-ocular lens is that it requires fitting within an artificial capsular bag that in turn is placed within the natural capsular bag The introduction into the artificial capsular bag of the eye is problematic and undesirable. Such introduction complicates a simple and efficient cataract procedure and increases the chance that the implant insertion will be difficult.

What is needed is an intra-ocular lens that can be directly fitted into the natural capsular bag or into the sulcus of the eye. This is a simpler procedure, assuring a greater success rate.

As with the aforementioned intra-ocular lens, the current inventive lens is activated by gravity and is constructed from materials, or a combination of materials, that provide a specific gravity greater than that of the aqueous humor. The lens is constructed with a plurality of haptics: two horizontal haptics and possibly an inferior haptic that might be needed to prevent the heavier-than-aqueous-fluid lens from de-centering inferiorly. The haptics are disposed within the lens in an articulated manner that permits movement of the lens between forward and rearward positions.

Another possible configuration is a lens with plate haptics similar to the Cummings lens but with an optic whose specific gravity is greater than aqueous so that it moves in response to gravity.

The lengths of the horizontal haptics are designed to be greater than the space available in the capsule or the sulcus, such that the only two stable positions of rest for the lens are the forward and the rearward positions, respectively. Two stops can be inserted to ensure that the intra-ocular lens achieves a precise forward and rearward position.

### DISCUSSION OF RELATED ART

In U.S. Pat. No. 4,512,040, issued to McClure on Apr. 23, 1985 for BIFOCAL INTRAOCULAR LENS, a bifocal, intra-ocular lens is shown that is activated by the transfer of liquid into and out of the refractive chamber, thus creating a bifocal effect.

In U.S. Pat. Nos. 5,496,366 and 6,197,059, issued to Cummings for ACCOMMODATING INTRAOCULAR LENS on Mar. 5, 1996 and Mar. 6, 2001, respectively, an intraocular lens is disclosed, the operation of which is based on fibrosis fusing the capsular bag of the human eye to the anterior capsular remnant. Ciliary muscle, not gravitational forces, is relied on to increase and decrease vitreous pressure in the eye. The stretching and relaxing of the ciliary muscle helps move the lens forward and backward to provide for accommodation.

In U.S. Pat. No. 4,254,509, issued to Tennant for ACCOMMODATING INTRAOCULAR IMPLANT on Mar. 10, 1981, an eye implant also relies on contraction of a ciliary body, not gravity, to move the lens anteriorly.

### SUMMARY OF THE INVENTION

In accordance with the present invention, there is provided an accommodating intra-ocular lens. The intra-ocular lens is gravity activated and constructed from materials that provide a specific gravity greater than that of the aqueous humor. The loop haptic lens is constructed with a plurality of haptics: two horizontal haptics and possibly an inferior haptic to prevent the heavier-than-aqueous-fluid lens from decentering inferiorly. The haptics, whether looped or plate, are disposed within the lens in an articulated manner that permits movement of the lens between forward and rearward positions. The lengths of the haptics are designed to be greater than the space available in the capsular bag or the sulcus, such that the only two stable positions of rest for the lens are the forward and the rearward positions, respectively. Two stops can be inserted to ensure that the intra-ocular lens achieves a precise forward and rearward position.

It is an object of the present invention to provide a gravity activated, accommodating, intra-ocular lens that can be directly fitted into the capsular bag or the sulcus of the eye.

### BRIEF DESCRIPTION OF THE DRAWINGS

A complete understanding of the present invention may be obtained by reference to the accompanying drawings, when considered in conjunction with the subsequent detailed description, in which:

FIG. 1 illustrates a schematic, anterior view of a looped haptic lens of this invention disposed within the capsular bag or the sulcus of the eye;

FIG. 2 depicts a schematic, top, in situ view of the lens shown in FIG. 1, positioned in its extreme respective forward and rearward positions;

FIG. 3 shows a schematic, sectional, top view of a first embodiment of the articulated lens and one possible hinged, looped haptic connection;

FIG. 4 shows a schematic, sectional, front view of a second embodiment of the articulated lens and one possible hinged, looped haptic connection;

FIG. 5 shows another embodiment of a looped haptic hinge where the haptic is inserted through a conical-shaped tunnel in the lens;

FIG. 6 shows the anterior view of the haptic post hinge where the radial post is inserted through a hole in the haptic to fasten the haptic to the lens, yet permit rotation of the haptic, in the conical-shaped tunnel, around the post that acts as an axis of rotation;

FIG. 7 shows the conical shape of the opening only in the anterior-posterior direction but cylindrical in the vertical direction;

FIG. 8 shows a plate haptic lens with hinges permitting anterior-posterior movement;

FIG. 9 shows how heavy buttons can be attached to the optics of the plate lens to increase the weight of the lens.

For purposes of brevity and clarity, like components and elements of the apparatus of this invention will bear the same designations or numbering throughout the figures.

### DESCRIPTION OF THE PREFERRED EMBODIMENT

Generally speaking, the invention features an accommodating, intra-ocular lens that is gravity activated and constructed from materials that provide a specific gravity greater than that of the aqueous humor. The lens is constructed with a plurality of haptics: two horizontal haptics and possibly an inferior haptic that prevents the heavier-than-aqueous-fluid lens from de-centering inferiorly. The haptics are disposed within the lens in an articulated manner that permits movement of the lens between forward and rearward positions.

Now referring to FIG. 1, the intra-ocular lens 10 of this invention is illustrated. The lens 10 is placed within the capsular bag or sulcus 12 by means of three haptics 14, all C-looped or J-looped at their respective distal ends. Two of the haptics 14 are horizontally disposed and one of the haptics 14 is inferiorly disposed therein.

Referring to FIG. 2, a superior view of the lens 10 is shown. The lens 10 is depicted in its respective forward and rearward positions as referenced by positions marked “A” and “B.” The haptics 14 are articulately connected to lens 10 at the pivot point 15. In the posterior position “B,” the lens 10 is 0.5 mm behind the mid-point “X.” In the anterior “A” position, it is 0.5 mm in front of the mid-point “X,” thus providing a total movement of 1.0 mm. The haptics 14 may also be spaced 120° apart from one another. The lens 10 is made from relatively dense materials that have a specific gravity greater than the aqueous humor. Such materials are well-known in the art.

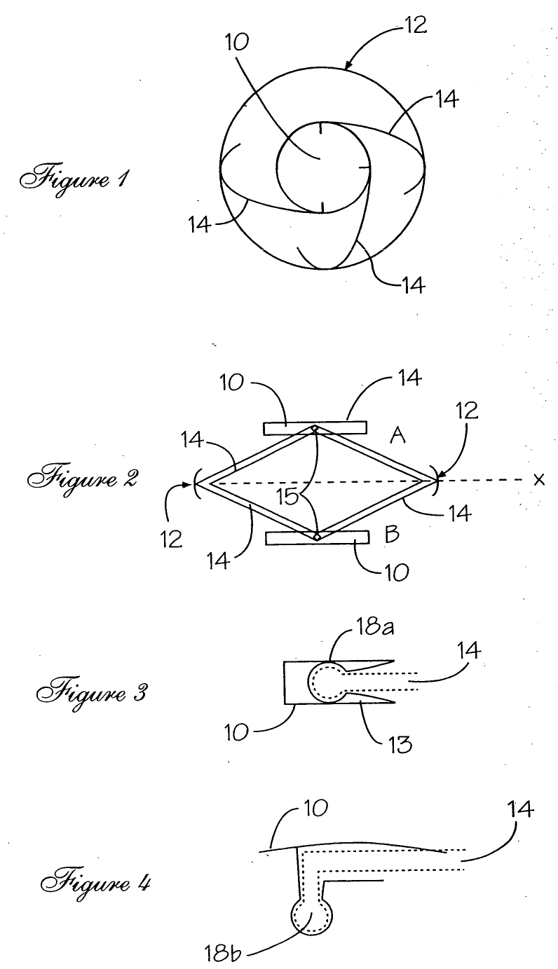

Referring to FIGS. 3 and 4, two sectional view embodiments are shown depicting how the haptics 14 are articulatively attached to the lens 10. The haptics 14 are connected to the lens 10 by means of ball-socket connections 18a and 18b. The respective ball-socket connections 18a and 18b are designed for limited movement between positions “A” and “B” of FIG. 2. The limits are provided by the wall of the groove 13 in the lens 10 through which the haptic 14 passes.

The lens 10 moves between positions “A” and “B” by means of gravity. When the individual (not shown) looks upwardly, the lens 10 moves to position “A,” and when the individual looks downwardly, the lens 10 moves to position “B.” The individual can also press posteriorly on the cornea, through the closed lid, in order to create a brief posterior force that moves the lens 10 backward to position “A.” An abrupt forward head movement can also impart a backward force to move lens 10 backward to the near focus position “A.”

Other forms of hinges with forward and backward stops are also possible alternatives to that shown in FIGS. 3 and 4

FIG. 5 shows another embodiment of a hinge where the haptic 14 is attached to the lens 10 by a vertical post 19 which acts as an axis around which the haptic 14 rotates. The rotation is limited by the walls of the tunnel 20 in the lens 10 which is conical on the anterior-posterior direction as shown. The opening is limited to the width of the haptic 14 in the vertical direction to limit vertical movement of the lens 10 in response to gravity and helps keep it centered. In addition, an inferior vertical haptic 14 may be needed, as in FIG. 1, to keep the lens 10 from sinking in response to gravity.

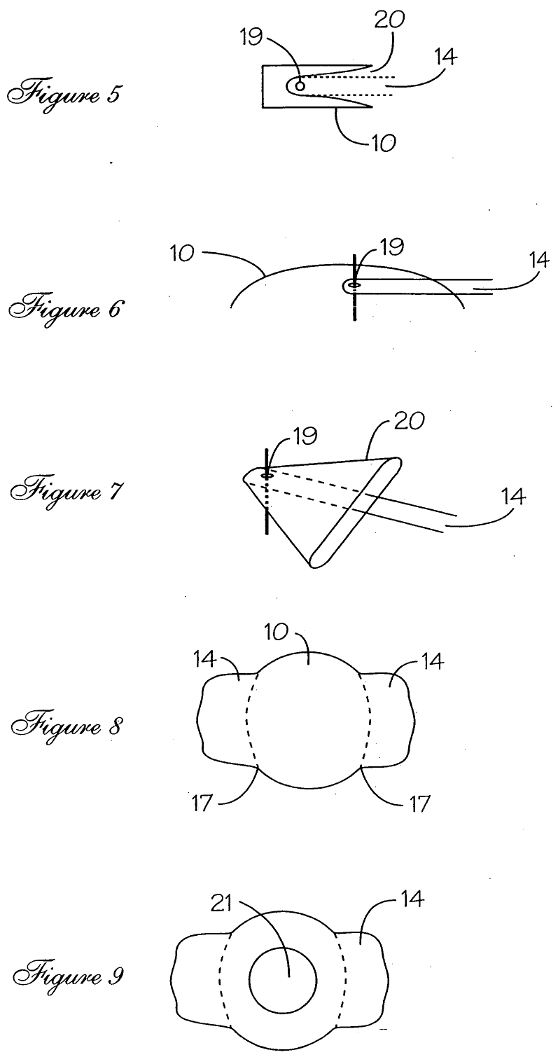

FIG. 6 depicts the anterior view of the haptic 14 post hinge where the radial post 19 is inserted through a hole in the haptic 14 to fasten it to the lens 10, yet permit rotation of the haptic 14 around the post 19 that acts as an axis of rotation. The hole in the lens 10 is cylindrical, not conical, when viewed from the anterior perspective in order to limit the vertical movement of the lens 10.

Referring to FIG. 7, a schematic of the tunnel 20 in the lens 10 (FIG. 6) is shown through which the haptic 14 is inserted into the opening where it is fastened by the radial positioned post-axle 19. The tunnel 20 is conical in the anterior-posterior direction and cylindrical in the vertical direction.

FIG. 8 shows a plate haptic lens 10 with hinges 17 permitting anterior-posterior movement with stops to the hinge 17 to limit movement of the optic to the desired anterior and posterior limits, similar to the Cummings lens but with an optic that has a specific gravity greater than the aqueous humor so that it moves in the anterior-posterior direction in response to gravity.

Now referring to FIG. 9, another embodiment of lens 10 is shown, with plate or loop haptics 14, hinged to permit anterior-posterior movement of the optic in response to gravity. A lens button 21 is shown having a high specific gravity that is a possible means to increase the weight of the optic to permit the lens 10 to respond to gravity by moving in the anterior-posterior direction. A similar button can be placed in the center of a loop haptic lens. The button can be round or horizontally oval to permit folding of the lens for insertion through a small opening yet provide a larger optic in the horizontal dimension.

In fact, no hinge may be necessary when the elasticity of the haptic 14 alone is sufficient to permit forward movement in response to gravity, and backward movement to a posterior position of rest when free of the influence of gravity.

Once the individual has completed his or her near vision task, the lens 10 can be returned to the posterior position “A” for distant vision focus by one of the following procedures:

a) squeezing the eyelids tightly;

b) applying digital pressure on the front of the cornea, through the eyelids;

c) moving the head abruptly anteriorly; or

d) gazing upwardly to allow gravity to move lens 10 to its posterior equilibrium position.

The relative ease of movement of the anterior positioned lens depends on the relative position of the equilibrium position x, as shown in FIG. 2. If the limits of the hinge are such that the forward position B is closer to x than is the posterior position A, it will be easier to move the lens back to the posterior position; however, it will be harder to move the lens forward from position A to position B. The position x is the position in which the haptics are in the same plane. This is an unstable position. Once the lens is moved beyond position x, the resultant anterior-posterior vectors force produced by tension in the haptics will move the lens to the stable end position determined by the limits of the haptic hinge. For example, in FIG. 2, if position A is 0.75 mm behind x and position B is 0.25 mm in front of x, it will be harder to move the lens to the anterior position but easier to return it by the above-described techniques once the need for near focus is complete.

Experimental calculations have determined that for a corneal power of 40D, an axial length of 24 mm, and a lens having a posterior position of 4 mm behind the cornea, a 21.4 D lens would be required to focus to infinity. Moving the lens forward 1 mm requires the eye to need +1.6 Diopters (instead of +3.00) to focus at 33 cm. This would be a significant improvement over the current art. A 2 mm forward movement of the lens would achieve 3.0 diopters of accommodation to permit focus at 33 mm without any additional correction.

Since other modifications and changes varied to fit particular operating requirements and environments will be apparent to those skilled in the art, the invention is not considered limited to the example chosen for purposes of disclosure and covers all changes and modifications which do not constitute departures from the true spirit and scope of this invention.

Having thus described the invention, what is desired to be protected by Letters Patent is presented in the subsequently appended claims.

What is claimed is:

1. An intra-ocular lens apparatus capable of being disposed within the capsular bag or sulcus of the eye, comprising:
a lens for movement by gravity between respective forward and rearward positions within said eye; and
a plurality of haptics, each articulately connected to said lens at one distal end and disposed within said capsular bag or sulcus on another distal end.

2. The intra-ocular lens in accordance with claim 1, wherein said plurality of haptics comprises two or three looped haptics or plate haptics.

3. The intra-ocular lens in accordance with claim 2, wherein said plurality of haptics comprises two horizontal haptics and possibly an inferior haptic.

4. The intra-ocular lens in accordance with claim 2, wherein said three haptics are spaced 120° apart from one another.

5. The intra-ocular lens in accordance with claim 1, wherein each of said plurality of haptics is connected to said lens by means of a ball-socket, a pin axle or a hinged plate haptic connection.

6. The intra-ocular lens in accordance with claim 1, wherein each of said plurality of haptics is connected to said lens by hinged means.

7. The intra-ocular lens in accordance with claim 1, wherein said haptics are elastic and retain said lens in a first position until a user looks downwardly and gravity moves said lens to a second position.

8. The intra-ocular lens in accordance with claim 1, wherein said lens comprises means for movement of approximately 1 mm between said forward and said rearward positions.

9. An accommodating intra-ocular lens apparatus capable of being disposed within the capsular bag or sulcus of the eye, comprising:
a lens for movement by gravity between respective forward and rearward focal positions within an eye, said lens having a specific gravity greater than an aqueous solution of said eye; and
a plurality of haptics, each articulately connected to said lens at one distal end and disposed within said capsular bag or sulcus on another distal end.

10. The intra-ocular lens in accordance with claim 9, wherein said plurality of haptics comprises two or three haptics.

11. The intra-ocular lens in accordance with claim 7, wherein said plurality of haptics comprises two horizontal haptics and possibly an inferior haptic.

12. The intra-ocular lens in accordance with claim 11, wherein said three haptics are spaced 120° apart from one another.

13. The intra-ocular lens in accordance with claim 9, wherein each of said plurality of haptics is connected to said lens by means of ball-socket connections or a pin axle connection.

14. The intra-ocular lens in accordance with claim 9, wherein each of said plurality of haptics is connected to said lens by hinged means with limits to the forward and backward movement of said lens.

15. The intra-ocular lens in accordance with claim 9, wherein said haptics are elastic and retain said lens in a first position until a user looks downwardly and gravity moves said lens to a second position.

16. The intra-ocular lens in accordance with claim 9, wherein said lens comprises means for movement of approximately 1 mm between said forward and said rearward positions.

17. The intra-ocular lens in accordance with claim 9 further comprising a lens button attached to or embedded in said lens, said lens button having a specific gravity greater than said specific gravity of said aqueous solution of said eye.

18. The intra-ocular lens in accordance with claim 17 further having an elongated button facilitating folding and insertion through a small opening in the eye.
